(19) United States
(12) Patent Application Publication
(10) Pub. No.: US 2025/0089640 A1
(43) Pub. Date: Mar. 20, 2025

(54) PHILODENDRON PLANT NAMED “ALOCASIA JACKLYN VARIEGATED”

(71) Applicant: Robert Roe, Umatilla, FL (US)

(72) Inventor: Robert Roe, Umatilla, FL (US)

(21) Appl. No.: 18/889,318

(22) Filed: Sep. 18, 2024

Related U.S. Application Data
(60) Provisional application No. 63/538,963, filed on Sep. 18, 2023.

(51) Int. Cl. A01H 6/10; A01H 5/12

(52) U.S. Cl. CPC A01H 6/10

(57) ABSTRACT

A new and distinct cultivar named “Alocasia Jacklyn Variegated”.

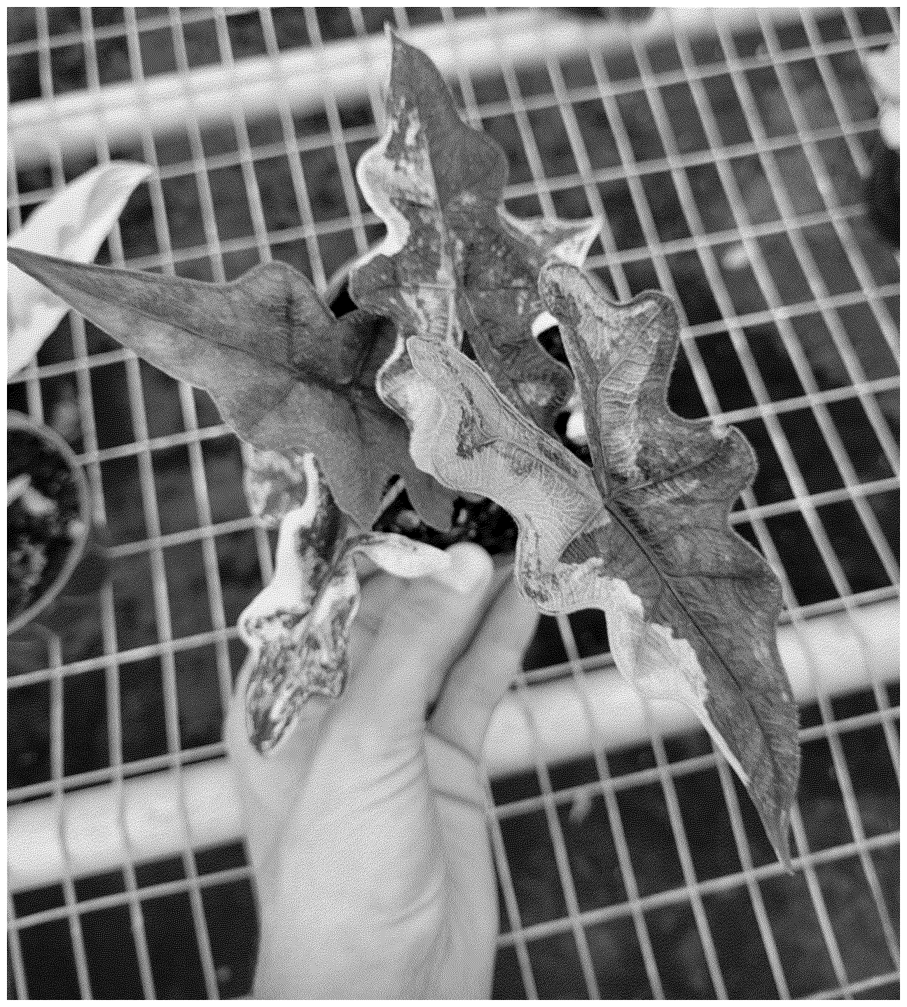

### CROSS-REFERENCE TO RELATED APPLICATIONS

The present application claims priority to U.S. Provisional Application No. 63/538,963 filed Sep. 18, 2023, titled “PHILODENDRON PLANT NAMED ALOCASIA JACKLYN VARIEGATED,” which is hereby incorporated by reference in its entirety.

### BACKGROUND

The present invention relates to a new and distinct cultivar of Alocasia Jacklyn Variegated plant.

The new Alocasia Jacklyn Variegated is a product of an accidental breeding by the Inventor in Umatilla, Florida.

### SUMMARY OF THE INVENTION

The new Alocasia Jacklyn Variegated has not been observed under all possible environmental conditions. The phenotype may vary somewhat with variations in environment such as temperature, light intensity, fertilizer rate, and/or irrigation amount and frequency without any variance in genotype.

The following traits have been repeatedly observed and are determined to be the unique characteristics of “Alocasia Jacklyn Variegated.” These characteristics in combination distinguish “Alocasia Jacklyn Variegated” as a new and distinct cultivar.
1. Plants of the new Alocasia Jacklyn Variegated have dark green leaves with white variegation that have black/green veins.
2. Leaves can grow 8-inches to 36 inches long.
3. Stems are long, thin, and dark and light brown.
4. The Alocasia Jacklyn Variegated may grow 2-6 feet tall.

### DETAILED BOTANICAL DESCRIPTION

The following observations, measurements, and comparisons describe plants grown in Umatilla, Florida, under a polycarbonate-covered greenhouse and conditions which closely approximate those used in general, standard commercial horticultural practice. Plants were grown in liquid granular fertilized in a dry tropical environment. Light level ranged from 2000 to 2,200 footcandles. Growth is possible between 2 to 6 feet tall.

What is claimed is:

1. A new and distinct cultivar named “Alocasia Jacklyn Variegated” as illustrated and described.
